(19) United States
(12) Patent Application Publication
(10) Pub. No.: US 2009/0246844 A1
(43) Pub. Date: Oct. 1, 2009

(54) Methods for the production of ethanol

(75) Inventors: Nikolai Khramtsov, Branford, CT (US); Alexander Amerik, Avon, CT (US); Steven A. Henck, Woodbridge, CT (US)

Correspondence Address: MINTZ LEVIN COHN FERRIS GLOVSKY & POPEO, ONE FINANCIAL CENTER, BOSTON, MA, 02111, US

(73) Assignee: Arbor Fuel Inc., Woodbridge, CT (US)

(21) Appl. No.: 12/386,858

(22) Filed: Apr. 23, 2009

Related U.S. Application Data
(63) Continuation-in-part of application No. PCT/US2008/012186, filed on Oct. 27, 2008.
(60) Provisional application No. 61/000,458, filed on Oct. 26, 2007.

(51) Int. Cl. C12P 7/06; C12N 1/19

(52) U.S. Cl. 435/161; 435/254.2; 435/254.21

(57) ABSTRACT

Embodiments of the present invention include methods for the production of ethanol, by a consolidated bioprocessing approach for the conversion of cellulosic material. According to some embodiments, recombinant microbial host cells are provided, preferably S. cerevisiae, that are capable of converting cellulosic material to ethanol and include cellulase genes. According to some embodiments, recombinant microbial host cells are provided, preferably S. cerevisiae, that are capable of converting hemicellulosic material to ethanol and include cellulase genes and at least one gene for the conversion of a pentose sugar.

### CROSS-REFERENCE TO RELATED APPLICATIONS

This application claims priority to International Patent Application No. PCT/US2008/012186, filed Oct. 27, 2008, which claims priority to U.S. Provisional Patent Application No. 61/000,458, filed Oct. 26, 2007, each of which is incorporated by reference into this disclosure in its entirety.

### FIELD OF THE INVENTION

This invention relates to methods and recombinant microorganisms for the production of ethanol by a consolidated bioprocessing approach for the conversion of cellulosic material to ethanol.

### BACKGROUND OF THE INVENTION

Biofuels are critical to securing energy infrastructures by providing alternative fuels, which will not only limit dependence on fossil fuels, but will also reduce detrimental carbon emissions generated and released into the atmosphere. Current efforts towards the implementation of biofuels have centered on ethanol production and its use.

One problem associated with current methods for the production of biofuels is the use of food crops, such as corn and sugar, as the starting material. For example, the use of cereal grains, such as corn, for ethanol production competes directly with the food supply, and thus has the unintended consequence of driving up source material costs.

An alternative to the use of food crops is cellulosic biomass, such as agricultural and forestry residues or municipal waste, such as waste paper or cardboard. Cellulosic biomass is more abundant and would be much less expensive to use than food stuffs. Unfortunately, the production of biofuels from cellulose and lignocellulose with current technologies is very difficult because of the complex molecular structure of lignocellulose. Current methods require multiple steps either utilizing acid treatment and neutralization, and subsequent treatment with exogenously produced enzymes to hydrolyze the cellulose to sugars, or alternatively syngas technologies are used. Syngas technologies use a considerable amount of heat to turn solid biomass into a mixture of gaseous vapors, and then use a catalyst to convert these gases into liquids, primarily mixed alcohols.

Cellulose is a stable polymer with a half-life about 5-8 million years for β-glucosidic bond cleavage at 25° C. (Wolfenden and Snider, 2001). The enzyme-driven cellulose biodegradation process is orders of magnitude faster, and is vital for returning carbon in sediments to the atmosphere (Zhang et al., 2006). The widely accepted mechanism for enzymatic cellulose hydrolysis involves synergistic actions of three different cellulases: endoglucanase, exoglucanase or cellobiohydrolase and β-glucosidase (Lynd et al., 2002). Endoglucanases (1,4-β-D-glucan 4-glucanohydrolases; EC 3.2.1.4) cleave intramolecular β-1,4-glucosidic linkages randomly. Exoglucanases (1,4-β-D-glucan cellobiohydrolases; EC 3.2.1.91) cleave the accessible ends of cellulose molecules to liberate cellobiose. β-glucosidases (β-glucoside glucohydrolases; EC 3.2.1.21) hydrolyze soluble cellobiose and other cellodextrins with a degree of polymerization up to 6 to produce glucose in the aqueous phase. The hydrolysis rates decrease markedly as the degree of substrate polymerization increases (Zhang and Lynd, 2004). Currently, most commercial cellulases are produced using Trichderma and Aspergillus species. The cellulose market is expected to expand dramatically when cellulases are used to hydrolyze pretreated cellulosic materials to sugars, which can be fermented to biofuels on a large scale. Genes encoding cellulases have been cloned from various bacteria, filamentous fungi and plants (Lynd et al., 2002). Several groups have expressed multiple cellulase enzymes in attempts to recreate a fully cellulolytic, fermentative system in Saccharomyces cerevisiae (van Zyl et al., 2007). Since S. cerevisiae lacks the enzymes that hydrolyze cellulose, three types of cellulases were codisplayed on the surface of the yeast cell wall. WO 2008/064314 describes yeast strains with four cellulases codisplayed on the surface of the yeast cell wall that can grow on and bind to cellulose. A yeast strain codisplaying on the cell wall surface endoglucanase II and cellobiohydrolase II from Trichoderma reesei (T. reesei), and Aspergillus aculeatus (A. aculeatus) β-glucosidase I was able to directly produce ethanol from amorphous cellulose with a yield of approximately 2.9 gram per liter (Fujita et al., 2004). Other groups expressed secreted forms of two cellulases: endoglucanase of T. reesei and β-glucosidase of Saccharomycopsis fibuligera (S. fibuligera), in combination in S. cerevisiae (Den Haan et al., 2007). The highest ethanol titer achieved was ˜1 gram per liter.

Accordingly, there is a need for more efficient methods and microorganisms for producing ethanol from cellulosic biomass.

### SUMMARY OF THE INVENTION

Recombinant microorganisms having an engineered pathway for the direct conversion of cellulosic material to ethanol are provided. Methods are provided for producing ethanol using these recombinant microorganisms. These methods integrate into a single microorganism or a stable mixed culture of microorganisms to increase production efficiency, the hydrolysis of cellulosic materials and subsequent fermentation of the resulting sugars to alcohol. More specifically, embodiments of the present invention integrate the production of alcohol, such as ethanol, with one or more of the following process steps:
1) Lignin removal from lignocellulose to allow enzymatic access to the cellulose and hemicellulose;
2) De-polymerization of cellulose and hemicellulose to soluble sugars; and
3) Fermentation of a mixed-sugar hydrolysate containing six-carbon (hexose) and five-carbon (pentose) sugars.

In one aspect, a recombinant microbial host organism is provided, preferably genetically modified S. cerevisiae, that is capable of converting cellulose to ethanol comprising a DNA molecule encoding at least one cellulase enzyme. In a preferred embodiment, the cellulase enzymes are selected from the group consisting of: endoglucanase II, cellobiohydrolase II, and β-glucosidase I.

In another aspect, a recombinant microbial host organism is provided, preferably genetically modified S. cerevisiae, that is capable of converting cellulose and hemicellulose to ethanol comprising: (1) a DNA molecule encoding at least one polypeptide involved in the fermentation of a pentose sugar, preferably xylose; (2) a DNA molecule encoding at least one cellulase enzyme. In a preferred embodiment, the at least one polypeptide involved in the fermentation of a pentose sugar is xylose isomerase.

It is contemplated that whenever appropriate, any embodiment of the present invention can be combined with one or more other embodiments of the present invention, even though the embodiments are described under different aspects of the present invention.

### BRIEF DESCRIPTION OF THE FIGURES

The invention can be more fully understood from the following detailed description, figure, and the accompanying sequence descriptions, which form a part of this application.

FIG. 1 shows a calibration curve for quantification of ethanol concentration using gas chromatography. A linear calibration curve was developed with a range of 1000 ppm to 0.8 ppm.

FIG. 2 shows ethanol production, as a function of time, from treated paper (top) and PASC (bottom) as the source of carbon, respectively. Three independent fermentations were performed with yeast strains Y1.C8 with three cell wall attached cellulases. Y1.B9, Y1.C1 and Y1.C2 contain 3 secreted cellulases; Y1.C9 is a control strain containing the same vectors without cellulases.

FIG. 3 shows ethanol production, as a function of time, from a 10 mL fermentation of PASC using the yeast strain Y1.C8.

FIG. 4 shows ethanol production, as a function of time, from a one liter fermentation of treated paper. The data is an average of three samples taken at each time point, and the error bars are ± one standard deviation.

FIG. 5 shows ethanol production, as a function of time, from cellulose and xylose as described in Example 7.

FIG. 6 shows ethanol production, as a function of time, from PASC using industrial yeast strains as described in Example 10.

### DETAILED DESCRIPTION OF THE INVENTION

Recombinant microorganisms are provided that have an engineered pathway for the direct conversion of cellulosic material to ethanol. Methods are also provided that integrate hydrolysis and fermentation into a single microorganism or a stable mixed culture of microorganisms to increase efficiency of production. More specifically, embodiments of the present invention integrate the production of alcohol, such as ethanol, with one or more of the following process steps:
1) Lignin removal from lignocellulose to allow enzymatic access to the cellulose and hemicellulose;
2) De-polymerization of cellulose and hemicellulose to soluble sugars; and
3) Fermentation of a mixed-sugar hydrolysate containing six-carbon (hexose) and five-carbon (pentose) sugars.

Unless defined otherwise, all technical and scientific terms used herein have the same meaning as commonly understood by one of ordinary skill in the art to which this invention belongs. In the case of conflict, the present specification will control. The following definitions and abbreviations are to be used for the interpretation of the claims and the specification.

The term “ethanol biosynthetic pathway” refers to a microbial pathway to produce ethanol.

The term “carbon substrate” refers to a carbon source capable of being metabolized by host organisms of the present invention, and particularly carbon sources selected from the group consisting of monosaccharides, oligosaccharides, polysaccharides, or mixtures thereof.

The term “gene” refers to a nucleic acid fragment that is capable of being expressed as a specific protein or RNA, optionally including regulatory sequences preceding (5′ non-coding sequences) and following (3′ non-coding sequences) the coding sequence. “Native gene” refers to a gene as naturally found in a host organism with its own regulatory sequences. “Chimeric gene” refers to any gene that is not a native gene, comprising regulatory and coding sequences that are not found together in the host organism. Accordingly, a chimeric gene may comprise regulatory sequences and coding sequences that are derived from different sources, or regulatory sequences and coding sequences derived from the same source, but arranged in a manner different than that found in that source. “Endogenous gene” refers to a native gene in its natural location in the genome of an organism. A “foreign gene” or “heterologous gene” refers to a gene not normally found in the host organism, but that is introduced into the host organism. Foreign genes can comprise native genes inserted into a non-native organism, or chimeric genes. It is also understood, that foreign genes encompass genes whose coding sequence has been modified to enhance its expression in a particular host, for example, codons can be substituted to reflect the preferred codon usage of the host.

As used herein the term “coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structures.

The term “promoter” refers to a DNA sequence capable of controlling the expression of a coding sequence or functional RNA (e.g., rRNA). A coding sequence is located downstream of a promoter sequence. Promoters may be derived in their entirety from the promoter of a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters.” It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of effecting the expression of that coding sequence (i.e., the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from nucleic acid fragments. Expression also refers to translation of mRNA into a polypeptide.

As used herein, the term “transformation” refers to the insertion of an exogenous nucleic acid into a cell, irrespective of the method used for the insertion, for example, lipofection, transduction, infection or electroporation. The exogenous nucleic acid can be maintained as a non-integrated vector, for example, a plasmid, or alternatively, can be integrated into the cell's genome. Host organisms containing the transformed nucleic acid fragments are referred to as “transgenic” or “recombinant” or “transformed” organisms.

The terms “plasmid”, “vector” and “cassette” refer to an extrachromosomal element often carrying genes which are not part of the central metabolism of the cell, and usually in the form of circular double-stranded DNA fragments. Such elements can be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation vector” refers to a vector or linear DNA fragment containing a foreign gene and having elements in addition to the foreign gene that facilitates transformation of a particular host cell. “Expression vector” refers to a vector containing a foreign gene and having elements in addition to the foreign gene that allow for enhanced expression of that gene in a foreign host.

As used herein, the term “maximum theoretical yield” is defined as the maximum amount of product (e.g., ethanol) that can be generated per total amount of substrate (e.g., glucose from cellulose) as dictated by the stoichiometry of the metabolic pathway used to make the product. For example, the maximum theoretical yield of ethanol starting with 261 g/L of PASC is 4 g/L. For example, the ethanol yield of 3.65 g/L observed in Example 6 would be expressed as 91.25% of the maximum theoretical yield. Alternatively, the maximum theoretical yield can be expressed in terms of grams of product generated per gram of substrate consumed.

As used herein, the term “industrial yeast strain” refers to a yeast strain that is suitable for use for the industrial production of ethanol (e.g., for use as a biofuel or as an alcoholic beverage, such as wine or beer). Industrial yeast strains include, but are not limited to, strains used for commercial and amateur winemaking and beer brewing. An industrial yeast strain will have one or more, preferably all, of the following characteristics: intrinsic tolerance to the ethanol, high temperature tolerance, high ethanol yield, and high growth rate. For example, in some embodiments, the industrial yeast strain will tolerate ethanol concentrations of greater than about 15%, greater than about 18%, greater than about 20% or greater than about 22%. In some embodiments, the industrial yeast strain will tolerate temperatures greater than 37° C. In some embodiments, the industrial yeast strain will tolerate temperatures of at least about 34° C., at least about 35° C., at least about 36° C., or at least about 37° C. In some embodiments, the industrial yeast strain will have a growth rate, such that the doubling time of the number of yeast cells is less than about 120 minutes, for example, between about 90 minutes and about 120 minutes, or about 100 minutes, or about 90 minutes, or less than about 90 minutes. In some embodiments, the industrial yeast strain will convert cellulose to ethanol at least about 80%, about 85%, about 90%, about 95% or about 99% or greater of the maximum theoretical yield. In some embodiments, the industrial yeast strain will produce ethanol from cellulose at a yield of at least about 3 g/L, about 3.2 g/L, about 3.5 g/L, about 3.8 g/L or about 4 g/L.

Standard molecular biology techniques used herein are well known in the art and are described by Sambrook J, Fritsch E F, Maniatis T. 1989. Molecular Cloning: A Laboratory Manual. Cold Spring Harbor Laboratory Press: Cold Spring Harbor, N.Y. Techniques for manipulation of S. cerevisiae used herein are well known in the art and are described in Methods in Yeast Genetics. 2005. Cold Spring Harbor Laboratory Press: Cold Spring Harbor, N.Y., and in Guthrie C, Fink G R, (Eds.). 2002. Methods in Enzymology, Volume 351, Guide to Yeast Genetics and Molecular and Cell Biology (Part C), Elsevier Academic Press, San Diego, Calif.

### Consolidated BioProcessing Approach

Consolidated bioprocessing (CBP), as used herein, is a processing strategy for cellulosic biomass which involves consolidating the production of alcohol, such as ethanol, with one or more of the following steps into a single process:
1) Lignin removal from lignocellulose to allow enzymatic access to the cellulose and hemicellulose;
2) Hydrolysis of cellulose and hemicellulose to soluble sugars; and
3) Fermentation of a mixed-sugar hydrolysate containing six-carbon (hexose) and five-carbon (pentose) sugars.
   1) Lignin Removal from Lignocellulose

Laccases are enzymes that catalyze the oxidation of a variety of phenolic compounds as well as diamines and aromatic amines. In fungi, laccases are involved in the degradation of lignocellulosic materials (Rodríguez-Couto and Toca-Herrera, 2006). Ligninolytic enzymes are notoriously difficult to express in non-fungal systems. However, some embodiments of the present invention use laccase genes to break down lignin and release the cellulose or hemicellulose. Other enzymes suitable for expression in yeast to breakdown lignin include: lignin peroxide and manganese-dependent peroxidase (Hammel and Cullen, 2008).

### 2) Depolymerization of Cellulose to Soluble Sugars

Enzymatic degradation of cellulose involves the coordinate action of at least three different types of cellulases. Such enzymes are given an Enzyme Commission (EC) designation according to the Nomenclature Committee of the International Union of Biochemistry and Molecular Biology (Eur. J. Biochem. 264: 607-609 and 610-650, 1999). Endo-β-(1,4)-glucanases (EC 3.2.1.4) cleave the cellulose strand randomly along its length, thus generating new chain ends. Exo-β-(1,4)-glucanases (EC 3.2.1.91) are processive enzymes and cleave cellobiosyl units (β-(1,4)-glucose dimers) from free ends of cellulose strands. Lastly, β-D-glucosidases (cellobiases: EC 3.2.1.21) hydrolyze cellobiose to glucose. All three of these general activities are required for efficient and complete hydrolysis of a polymer such as cellulose to a subunit, such as the simple sugar, glucose. Cellulose degrading yeast strains can be made, for example in S. cerevisiae, by codisplaying on the cell surface or by coexpressing secreted forms cellulolytic enzymes from the filamentous fungi T. reesei and A. aculeatus.
3) Fermentation of a Mixed-Sugar Hydrolysate Containing Six-Carbon (hexose) and Five-Carbon (Pentose) Sugars

One of the most effective ethanol-producing yeasts, S. cerevisiae, has several advantages such as high ethanol production from hexoses and high tolerance to ethanol and other inhibitory compounds in the acid hydrolysates of lignocellulose biomass. However, because standard, wild-type, strains of this yeast cannot utilize pentoses, such as xylose, and celloligosaccharides (two to six glucose units), fermentation from a lignocellulose hydrolysate will not be completely efficient. According to certain embodiments of the present invention, a recombinant yeast strain is provided that can ferment xylose and cellooligosaccharides by integrating a gene for the intercellular expression of xylose isomerase from Piromyces sp. and a gene for displaying β-glucosidase from A. acleatus. According to some embodiments of the present invention, a recombinant yeast strain is provided that can ferment xylose and cellooligosaccharides by integrating genes for the intercellular expression of xylose reductase and xylitol dehydrogenase from Pichia stipitis (P. stipitis) and a gene for displaying β-glucosidase from A. acleatus.

### Microbial Hosts for Ethanol Production

Microbial hosts for ethanol production may be selected from bacteria, cyanobacteria, filamentous fungi and yeasts. The microbial hosts selected for the industrial production of ethanol, i.e., industrial strains, are preferably tolerant to ethanol and should be able to convert carbohydrates to ethanol with a high yield. Suitable microbial hosts include hosts with one or more, preferably all, of the following characteristics: intrinsic tolerance to the product, high temperature tolerance, high rate of carbon substrate utilization, efficiency in converting the carbon substrate to product, high growth rate, availability of genetic tools for gene manipulation, and the ability to generate stable chromosomal alterations.

The ability to genetically modify the host is useful for the production of a recombinant microorganism. The mode of gene transfer technology may be any method known in the art, such as by electroporation, conjugation, chemical transformation or transduction or transformation. A broad range of host conjugative plasmids and drug resistance markers are available and known to one of skill in the art.

In preferred embodiments, heterologous genes will be stably integrated into the host's genomic DNA. Stably integrating the heterologous genes will create stable transformants, which avoid the requirement for constant selection pressure. In this approach, the need for auxotrophic or antibiotic markers is eliminated. In certain preferred embodiments, multiple copies of the heterologous genes will stably integrate into the host's genomic DNA.

Additionally, the production host should be amenable to chemical mutagenesis or transposon-induced mutagenesis so that mutations to improve intrinsic ethanol tolerance may be obtained. Genome shuffling can also be used to develop and improve complex phenotypes. For example, genome shuffling can be used to increase ethanol production and tolerance in S. cerevisiae. Using yeast sexual and asexual reproduction, mutant diploid cells can be shuffled through highly efficient sporulation and adequate crossing among the resultant haploid cells, followed by selection on special selection plates. The selected strain obtained after several rounds genome shuffling not only can improve resistance to ethanol, but also can increase ethanol yield.

Suitable microbial hosts for the production of ethanol include, but are not limited to, members of the genera Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula and Saccharomyces. Preferred hosts include: Escherichia coli, Alcaligenes eutrophus, Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis, Saccharomyces carlsburgenesis and Saccharomyces cerevisiae. A preferred microbial host is a Saccharomyces species, for example, Saccharomyces bayanus, Saccharomyces carlsburgenesis or Saccharomyces cerevisiae. A particularly preferred microbial host is Saccharomyces cerevisiae.

### Construction of Production Host

Recombinant organisms containing the genes encoding the enzymatic pathway for the conversion of cellulose substrate to ethanol are constructed using techniques well known in the art. Methods of obtaining desired genes from a bacterial genome are common and well known in the art of molecular biology. For example, if the sequence of the gene is known, suitable genomic libraries may be created by restriction endonuclease digestion and may be screened with probes complementary to the desired gene sequence. Once the sequence is isolated, the DNA can be amplified using standard primer-directed amplification methods such as polymerase chain reaction (PCR; U.S. Pat. No. 4,683,202) to obtain amounts of DNA suitable for transformation using appropriate vectors. Alternatively, the gene sequence can be amplified directly using standard primer-directed amplification methods such as PCR using genomic DNA as a template. Additionally, the gene can be chemically synthesized.

Once the relevant pathway genes are identified and isolated they can be ligated to the vector DNA and transformed into suitable expression hosts by means well known in the art. Vectors and cassettes useful for the transformation of a variety of host cells are common and commercially available from companies such as EPICENTRE® (Madison, Wis.), Invitrogen Corp. (Carlsbad, Calif.), Stratagene (La Jolla, Calif.), and New England Biolabs, Inc. (Beverly, Mass.). Typically the vectors and cassettes contain sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. Both control regions may be derived from genes homologous to the transformed host cell, although it is to be understood that such control regions may also be derived from genes that are not native to the specific species chosen as a production host.

Initiation control regions or promoters, which are useful to drive expression of the relevant pathway coding regions in the desired host cell are numerous and familiar to those skilled in the art. Virtually any promoter capable of driving these genetic elements is suitable for the present invention. Promoters useful for expression in Saccharomyces include, but are not limited to CYC1, HIS3, GAL1, GAL10, ADH1, PGK, PHO5, GAPDH, ADC1, TRP1, URA3, LEU2, ENO, TP1, CUP1, FBA, GPD and GPM. A preferred promoter for expression in Saccharomyces is the GAPDH promoter from S. cerevisiae.

Termination control regions may also be derived from various genes native to the preferred hosts. Optionally, a termination site may be unnecessary; however, it is most preferred if included. Termination control regions useful for expression in Saccharomyces include, but are not limited to the CYC1, FBAt, GPDt, GPMt, ERG10t, GALt1 and ADH1 terminators. A preferred terminator for use in Saccharomyces is the CYC1 terminator from S. cerevisiae.

All sequence citations, references, patents, patent applications or other documents cited are hereby incorporated by reference.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Example 1

### Construction of Expression Plasmids Encoding Cellulase Genes

Expression constructs encoding cellulases for codisplay on the yeast cell wall surface were constructed by fusing cellulase genes with the DNA encoding the secretion signal sequence of glucoamylase from Rhizopus oryzae. The secretion signal is responsible for delivery of the cellulase to the cell wall. The gene, encoding the C-terminal half of S. cerevisiae α-agglutinin was linked to the 3′-end of the cellulase. The α-agglutinin part of the recombinant protein allows for the attachment to the cell wall. Furthermore, all three cellulases were also expressed in secreted soluble forms that are not attached to the cell wall. Expression constructs for secreted forms lacked the α-agglutinin portion.

DNA sequences of cellulase genes are known, and the following genes were used: T. reesei endoglucanase II (GenBank accession number DQ178347); T. reesei cellobiohyrdolase II (GenBank accession number M55080) and A. aculeatus β-glucosidase I (GenBank accession number D64088). The cellulase DNA constructs were commercially synthesized by Blue Heron Bio using their GeneMaker® synthesis platform. Unique restriction endonuclease sites were added to the sequences to facilitate subcloning into expression vectors. Several restriction sites were removed from coding sequences via one nucleotide substitutions that did not change the amino acid sequence.

The cellulase DNA constructs were cloned into the Blue Heron pUC119 vector. The sequences of the vector inserts are shown below:

pUC119-AF101 (cellobiohydrolase II (CBHII)
construct):

(SEQ ID NO: 1)

AAGCTTGCATGCAGTTTATCATTATCAATACTCGCCATTTCAAAGAATAC
GTAAATAATTAATAGTAGTGATTTTCCTAACTTTATTTAGTCAAAAAATT
AGCCTTTTAATTCTGCTGTAACCCGTACATGCCCAAAATAGGGGGCGGGT
TACACAGAATATATAACATCGTAGGTGTCTGGGTGAACAGTTTATTCCTG
GCATCCACTAAATATAATGGAGCCCGCTTTTTAAGCTGGCATCCAGAAAA
AAAAAGAATCCCAGCACCAAAATATTGTTTTCTTCACCAACCATCAGTTC
ATAGGTCCATTCTCTTAGCGCAACTACAGAGAACAGGGGCACAAACAGGC
AAAAAACGGGCACAACCTCAATGGAGTGATGCAACCTGCCTGGAGTAAAT
GATGACACAAGGCAATTGACCCACGCATGTATCTATCTCATTTTCTTACA
CCTTCTATTACCTTCTGCTCTCTCTGATTTGGAAAAAGCTGAAAAAAAAG
GTTGAAACCAGTTCCCTGAAATTATTCCCCTACTTGACTAATAAGTATAT
AAAGACGGTAGGTATTGATTGTAATTCTGTAAATCTATTTCTTAAACTTC
TTAAATTCTACTTTTATAGTTAGTCTTTTTTTTAGTTTTAAAACACCAGA
ACTTAGTTTCGACGGATCTGCAGGTCGACATGCAACTGTTCAATTTGCCA
TTGAAAGTTTCATTCTTTCTCGTCCTCTCTTACTTTTCTTTGCTCGTTTC
TGCTGACTACAAGGACGATGACGACAAATCTAGACAGGCTTGCTCAAGCG
TCTGGGGCCAATGTGGTGGCCAGAATTGGTCGGGTCCGACTTGCTGTGCT
TCCGGAAGCACATGCGTCTACTCCAACGACTATTACTCCCAGTGTCTTCC
CGGCGCTGCAAGCTCAAGCTCGTCCACGCGCGCCGCATCGACGACTTCAC
GAGTATCCCCCACAACATCCCGGTCGAGTTCCGCGACGCCTCCACCTGGT
TCTACTACTACCAGAGTACCTCCAGTCGGATCGGGAACCGCTACGTATTC
AGGCAACCCTTTTGTTGGGGTCACTCCTTGGGCCAATGCATATTACGCCT
CTGAAGTTAGCAGCCTCGCTATTCCTAGCTTGACTGGAGCCATGGCCACT
GCCGCAGCAGCTGTCGCAAAGGTTCCCTCTTTTATGTGGCTAGATACTCT
TGACAAGACCCCTCTCATGGAGCAAACCTTGGCCGACATCCGCACCGCCA
ACAAGAATGGCGGTAACTATGCCGGACAGTTTGTGGTGTATGACTTGCCG
GATCGCGATTGCGCTGCCCTTGCCTCGAATGGCGAATACTCTATTGCCGA
TGGTGGCGTCGCCAAATATAAGAACTATATCGACACCATTCGTCAAATTG
TCGTGGAATATTCCGATATCCGGACCCTCCTGGTTATTGAGCCTGACTCT
CTTGCCAACCTGGTGACCAACCTCGGTACTCCAAAGTGTGCCAATGCTCA
GTCAGCCTACCTTGAGTGCATCAACTACGCCGTCACACAGCTGAACCTTC
CAAATGTTGCGATGTATTTGGACGCTGGCCATGCAGGATGGCTTGGCTGG
CCGGCAAACCAAGACCCGGCCGCTCAGCTATTTGCAAATGTTTACAAGAA
TGCATCGTCTCCGAGAGCACTTCGCGGATTGGCAACCAATGTCGCCAACT
ACAACGGGTGGAACATTACCAGCCCCCCATCGTACACGCAAGGCAACGCT
GTCTACAACGAGAAGCTGTACATCCACGCTATTGGACGTCTTCTTGCCAA
TCACGGCTGGTCCAACGCCTTCTTCATCACTGATCAAGGTCGATCGGGAA
AGCAGCCTACCGGACAGCAACAGTGGGGAGACTGGTGCAATGTGATCGGC
ACCGGATTTGGTATTCGCCCATCCGCAAACACTGGGGACTCGTTGCTGGA
TTCGTTTGTCTGGGTCAAGCCAGGCGGCGAGTGTGACGGCACCAGCGACA
GCAGTGCGCCACGATTTGACTCCCACTGTGCGCTCCCAGATGCCTTGCAA
CCGGCGCCTCAAGCTGGTGCTTGGTTCCAAGCCTACTTTGTGCAGCTTCT
CACAAACGCAAACCCATCGTTCCTGGGATCCAGCGCCAAAAGCTCTTTTA
TCTCAACCACTACTACTGATTTAACAAGTATAAACACTAGTGCGTATTCC
ACTGGTTCCATTTCCACAGTAGAAACAGGCAATCGAACTACATCAGAAGT
GATCAGTCATGTGGTGACTACCAGCACAAAACTGTCTCCAACTGCTACTA
CCAGCCTGACAATTGCACAAACCAGTATCTATTCTACTGACTCAAATATC
ACAGTAGGAACAGATATTCACACCACATCAGAAGTGATTAGTGATGTGGA
AACCATTAGCAGAGAAACAGCTTCGACCGTTGTAGCCGCTCCAACCTCAA
CAACTGGATGGACAGGCGCTATGAATACTTACATCCCGCAATTTACATCC
TCTTCTTTCGCAACAATCAACAGCACACCAATAATCTCTTCATCAGCAGT
ATTTGAAACCTCAGATGCTTCAATTGTCAATGTGCACACTGAAAATATCA
CGAATACTGCTGCTGTTCCATCTGAAGAGCCCACTTTTGTAAATGCCACG
AGAAACTCCTTAAATTCCTTTTGCAGCAGCAAACAGCCATCCAGTCCCTC
ATCTTATACGTCTTCCCCACTCGTATCGTCCCTCTCCGTAAGCAAAACAT
TACTAAGCACCAGTTTTACGCCTTCTGTGCCAACATCTAATACATATATC
AAAACGGAAAATACGGGTTACTTTGAGCACACGGCTTTGACAACATCTTC
AGTTGGCCTTAATTCTTTTAGTGAAACAGCACTCTCATCTCAGGGAACGA
AAATTGACACCTTTTTAGTGTCATCCTTGATCGCATATCCTTCTTCTGCA
TCAGGAAGCCAATTGTCCGGTATCCAACAGAATTTCACATCAACTTCTCT
CATGATTTCAACCTATGAAGGTAAAGCGTCTATATTTTTCTCAGCTGAAC
TCGGTTCGATCATTTTTCTGCTTTTGTCGTACCTGCTATTCTAACCCGGG
TACCTCATGTAATTAGTTATGTCACGCTTACATTCACGCCCTCCCCCCAC
ATCCGCTCTAACCGAAAAGGAAGGAGTTAGACAACCTGAAGTCTAGGTCC
CTATTTATTTTTTTATAGTTATGTTAGTATTAAGAACGTTATTTATATTT
CAAATTTTTCTTTTTTTTCTGTACAGACGCGTGTACGCATGTAACATTAT
ACTGAAAACCTTGCTTGAGAAGGTTTTGGGACGCTCGAAGGCTTTAATTT
GCGGCCGAGCTCGAATTC

### Where Nucleotides:

1 to 12 are HindIII and SphI restriction sites;
13 to 667 is the constitutive GAPDH (glyceraldehyde 3-phosphate dehydrogenase) promoter (GenBank accession number DQ019861) from S. cerevisiae;
668 to 679 are PstI and SalI restriction sites;
680 to 754 is ATG and secretion signal from the R. oryzae glucoamylase gene (GenBank accession number D00049);
755 to 778 is a FLAG tag;
779 to 784 is a XbaI restriction site;
785 to 2125 is mature cellobiohydrolase II (CBHII) from T. reesi (GenBank accession number M55080), with the following nucleotide changes introduced (numbering according to the M55080 DNA sequence): A75G, G225A, T237A, C267T, T441C, G561C, T957A, and G1345C;
2126 to 2131 is a BamHI restriction site;
2132 to 3094 is the α-agglutinin 3′-gene portion with STOP codon (GenBank accession number AAA34417 or M28164), with the following nucleotide changes introduced (numbering according to the M28164 DNA sequence): T1422A, T1887C, and A2265G;
3095 to 3104 are SmaI-KpnI restriction sites;
3105 to 3356 is the CYC1 (cytochrome C) terminator (GenBank accession number EF210199); and
3357 to 3368 are SacI-EcoRI restriction sites.

pUC119-AF102 (β-glucosidase I (BGLI) construct):

(SEQ ID NO: 2)

TCTAGAGATGAACTGGCGTTCTCTCCTCCTTTCTACCCCTCTCCGTGGGC
CAATGGCCAGGGAGAGTGGGCGGAAGCCTACCAGCGTGCAGTGGCCATTG
TATCCCAGATGACTCTGGATGAGAAGGTCAACCTGACCACCGGAACTGGA
TGGGAGCTGGAGAAGTGCGTCGGTCAGACTGGTGGTGTCCCAAGACTGAA
CATCGGTGGCATGTGTCTTCAGGACAGTCCCTTGGGTATTCGTGATAGTG
ACTACAATTCGGCTTTCCCTGCTGGTGTCAACGTTGCTGCGACATGGGAC
AAGAACCTTGCTTATCTACGTGGTCAGGCTATGGGTCAAGAGTTCAGTGA
CAAAGGAATTGATGTTCAATTGGGACCGGCCGCGGGTCCCCTCGGCAGGA
GCCCTGATGGAGGTCGCAACTGGGAAGGTTTCTCTCCAGACCCGGCTCTT
ACTGGTGTGCTCTTTGCGGAGACGATTAAGGGTATTCAAGACGCTGGTGT
CGTGGCGACAGCCAAGCATTACATTCTCAATGAGCAAGAGCATTTCCGCC
AGGTCGCAGAGGCTGCGGGCTACGGATTCAATATCTCCGACACGATCAGC
TCTAACGTTGATGACAAGACCATTCATGAAATGTACCTCTGGCCCTTCGC
GGATGCCGTTCGCGCCGGCGTTGGCGCCATCATGTGTTCCTACAACCAGA
TCAACAACAGCTACGGTTGCCAGAACAGTTACACTCTGAACAAACTTCTG
AAGGCCGAACTCGGCTTCCAGGGCTTTGTGATGTCTGACTGGGGTGCTCA
CCACAGTGGTGTTGGCTCTGCTTTGGCCGGCTTGGATATGTCAATGCCTG
GCGATATCACCTTCGATTCTGCCACTAGTTTCTGGGGAACCAACCTGACC
ATTGCTGTGCTCAACGGAACCGTCCCGCAGTGGCGCGTTGACGACATGGC
TGTCCGTATCATGGCTGCCTACTACAAGGTTGGCCGCGACCGCCTGTACC
AGCCGCCTAACTTCAGCTCCTGGACTCGCGATGAATACGGCTTCAAGTAT
TTCTACCCCCAGGAAGGGCCCTATGAGAAGGTCAATCACTTTGTCAATGT
GCAGCGCAACCACAGCGAGGTTATTCGCAAGTTGGGAGCAGACAGTACTG
TTCTACTGAAGAACAACAATGCCCTGCCGCTGACCGGAAAGGAGCGCAAA
GTTGCGATCCTGGGTGAAGATGCTGGTTCCAACTCGTACGGTGCCAATGG
CTGCTCTGACCGTGGCTGTGACAACGGTACTCTTGCTATGGCTTGGGGTA
GCGGCACTGCCGAATTTCCATATCTCGTGACCCCTGAGCAGGCTATTCAA
GCCGAGGTGCTCAAGCATAAGGGCAGCGTCTACGCCATCACGGACAACTG
GGCGCTGAGCCAGGTGGAGACCCTCGCTAAACAAGCCAGTGTCTCTCTTG
TATTTGTCAACTCGGACGCGGGAGAGGGCTATATCTCCGTGGACGGAAAC
GAGGGCGACCGCAACAACCTCACCCTCTGGAAGAACGGCGACAACCTCAT
CAAGGCTGCTGCAAACAACTGCAACAACACCATCGTTGTCATCCACTCCG
TTGGACCTGTTTTGGTTGACGAGTGGTATGACCACCCCAACGTTACTGCC
ATCCTCTGGGCGGGCTTGCCTGGCCAGGAGTCTGGCAACTCCTTGGCTGA
CGTGCTCTACGGCCGCGTCAACCCAGGCGCCAAATCTCCATTCACCTGGG
GCAAGACGAGGGAGGCGTACGGGGATTACCTTGTCCGTGAACTCAACAAC
GGCAACGGAGCACCCCAAGATGATTTCTCGGAAGGTGTTTTCATTGACTA
CCGCGGATTCGACAAGCGCAATGAGACCCCGATCTACGAGTTCGGACATG
GTCTGAGCTACACCACTTTCAACTACTCTGGCCTTCACATCCAGGTTCTC
AACGCTTCCTCCAACGCTCAAGTAGCCACTGAGACTGGCGCCGCTCCCAC
CTTCGGACAAGTCGGCAATGCCTCTGACTACGTGTACCCTGAGGGATTGA
CCAGAATCAGCAAGTTCATCTATCCCTGGCTTAATTCCACAGACCTGAAG
GCCTCATCTGGCGACCCGTACTATGGAGTCGACACCGCGGAGCACGTGCC
CGAGGGTGCTACTGATGGCTCTCCGCAGCCCGTTCTGCCTGCCGGTGGTG
GCTCTGGTGGTAACCCGCGCCTCTACGATGAGTTGATCCGTGTTTCGGTG
ACAGTCAAGAACACTGGTCGTGTTGCCGGTGATGCTGTGCCTCAATTGTA
TGTTTCCCTTGGTGGACCCAATGAGCCCAAGGTTGTGTTGCGCAAATTCG
ACCGCCTCACCCTCAAGCCCTCCGAGGAGACGGTGTGGACGACTACCCTG
ACCCGCCGCGATCTGTCTAACTGGGACGTTGCGGCTCAGGACTGGGTCAT
CACTTCTTACCCGAAGAAGGTCCATGTTGGTAGCTCTTCGCGTCAGCTGC
CCCTTCACGCGGCGCTCCCGAAGGTGCAAGGATCCTAAGGTACC

### Where Nucleotides:

1 to 6 is a XbaI restriction site;
7 to 2529 is the mature β-glucosidase I from A. aculeatus (GenBank accession numbers D64088 or BAA10968), with the following nucleotide changes introduced (numbering according to the D64088 DNA sequence): A398T, G905A, G920A, T1049A, and T1079A;
A1388T; C1478T; G1886A, G1952A, T1973A;
2530 to 2535 is a BamHI restriction site;
2536 to 2538 is a TAA STOP codon; and
2539 to 2544 is a KpnI restriction site.

pUC119-AF103 (endoglucanase (EGII) construct):

(SEQ ID NO: 3)

TCTAGACAGCAGACTGTCTGGGGCCAGTGTGGAGGTATTGGTTGGAGCGG
ACCTACGAATTGTGCTCCTGGCTCAGCTTGTTCGACCCTCAATCCTTATT
ATGCGCAATGTATTCCGGGAGCCACTACTATCACCACTTCGACCCGGCCA
CCATCCGGTCCAACCACCACCACCAGGGCTACCTCAACAAGCTCATCAAC
TCCACCCACTAGCTCTGGGGTCCGATTTGCCGGCGTTAACATCGCGGGTT
TTGACTTTGGCTGTACCACAGATGGCACTTGCGTTACCTCGAAGGTTTAT
CCTCCGTTGAAGAACTTCACCGGCTCAAACAACTACCCCGATGGCATCGG
CCAGATGCAGCACTTCGTCAACGAGGACGGGATGACTATTTTCCGCTTAC
CTGTCGGATGGCAGTACCTCGTCAACAACAATTTGGGCGGCAATCTTGAT
TCCACGAGCATTTCCAAGTATGATCAGCTTGTTCAGGGGTGCCTGTCTCT
GGGCGCATACTGCATCGTTGACATCCACAATTATGCTCGATGGAACGGTG
GGATCATTGGTCAGGGCGGCCCTACTAATGCTCAATTCACGAGCCTTTGG
TCGCAGTTGGCATCAAAGTACGCATCTCAGTCGAGGGTGTGGTTCGGCAT
CATGAATGAGCCCCACGACGTGAACATCAACACCTGGGCTGCCACGGTCC
AAGAGGTTGTAACCGCAATCCGCAACGCTGGTGCTACGTCGCAATTCATC
TCTTTGCCTGGAAATGATTGGCAATCTGCTGGGGCTTTCATATCCGATGG
CAGTGCAGCCGCCCTGTCTCAAGTCACGAACCCGGATGGGTCAACAACGA
ATCTGATTTTTGACGTGCACAAATACTTGGACTCAGACAACTCCGGTACT
CACGCCGAATGTACTACAAATAACATTGACGGCGCCTTTTCTCCGCTTGC
CACTTGGCTCCGACAGAACAATCGCCAGGCTATCCTGACAGAAACCGGTG
GTGGCAACGTTCAGTCCTGCATACAAGACATGTGCCAGCAAATCCAATAT
CTCAACCAGAACTCAGATGTCTATCTTGGCTATGTTGGTTGGGGTGCCGG
ATCATTTGATAGCACGTATGTCCTGACGGAAACACCGACTGGCAGTGGTA
ACTCATGGACGGACACATCCTTGGTCAGCTCGTGTCTCGCAAGAAAGGGA
TCCTAAGGTACC

### Where Nucleotides:

1 to 6 is a XbaI restriction site;
7 to 1197 is the mature endoglucanase from T. reesei (GenBank accession numbers
DQ178347 or P07982), with the following nucleotide changes introduced (numbering according to the DQ178347 DNA sequence): G267T and C576T;
1198 to 1203 is a BamHI restriction site;
1204 to 1206 is a TAA STOP codon; and
1207 to 1212 is a KpnI restriction site.

Each of the above plasmids was used to create corresponding expression plasmids for cell wall attached cellulases. The cellulase expression vectors were generated using the pUC19-based yeast-E. coli shuttle vectors YEplac112, YEplac195 and YEplac181 (containing selectable markers TRP1, URA3 and LEU2, respectively) that have the yeast 2μ plasmid DNA replication origin (Gietz and Sugino, 1988). These shuttle vectors are high copy, small size vectors that can efficiently transform S. cerevisiae. For cell wall attached CBHII, pUC119-AF101 DNA was digested with HindIII-EcoRI and the 3370 bp DNA fragment was gel purified. The purified DNA fragment was ligated into the HindIII-EcoRI digested vectors YEplac112, YEplac181 and YEplac195, to generate YEplac112-AF101-at, YEplac181-AF101-at and YEplac195-AF101-at, respectively. For cell wall attached BGLI, pUC119-AF102 DNA was digested with XbaI-BamHI and the 2520 bp DNA fragment was gel purified. The purified DNA fragment was ligated into the XbaI-BamHI digested YEplac181-AF101-at vector, to generate YEplac181-AF102-at. For cell wall attached EGII, pUC119-AF103 DNA was digested with XbaI-BamHI and the 1212 bp DNA fragment was gel purified. The purified DNA fragment was ligated into the XbaI-BamHI digested YEplac112-AF101-at vector, to generate YEplac112-AF103-at.

Expression plasmids for secreted cellulases were also generated. For secreted BGLI, pUC119-AF102 DNA was digested with XbaI-KpnI and the 2530 bp DNA fragment was gel purified. The purified DNA fragment was ligated into XbaI-KpnI digested vectors YEplac181-AF101-at and YEplac195-AF101, to generate YEplac181-AF102-sec and YEplac195-AF102-sec, respectively. For secreted EGII, pUC119-AF103 DNA was digested with XbaI-KpnI and the 1212 bp DNA fragment was gel purified. The purified DNA fragment was ligated into the XbaI-KpnI digested YEplac112-AF103-at vector, to generate YEplac112-AF103-sec. For secreted CBHII, pUC119-AF101 DNA was digested with XbaI-BamHI and the 1341 bp DNA fragment was gel purified. The purified DNA fragment was ligated into the XbaI-BamHI digested YEplac195-AF102-sec, to generate YEplac195-AF101-sec.

### Example 2

### Transformation of S. cerevisiae and Transformant Selection

Derivatives of yeast strains AFY1 (MATα his3-Δ200 leu2-3, 112ura3-52 lys2-801 trp1-1) and AFY2 (MATa his3-Δ200 leu2-3,112 ura3-52 lys2-801 trp1-1) (Table 1) were used. These strains can be transformed with up to five plasmids carrying different selection markers. Transformation with the expression plasmids were performed with a lithium acetate method. Co-transformation with up to 3 plasmids was performed and the Trp⁺Ura⁺Leu⁺ colonies containing plasmids encoding cellulases were selected.

The yeast transformation procedure used was a slightly modified version of the protocol described in Ausubel et al., (2002). Cells from an overnight culture were resuspended in 50 mL YPD (start OD₆₀₀ of 0.2) and grown to an OD₆₀₀ of 0.5-0.7. The cells were harvested by centrifugation (1,500 g, 5 min) and resuspended in 20 mL sterile distilled water. The cells were harvested by centrifugation and resuspended in 1.5 mL of freshly prepared sterile TE/LiOAc (prepared from 10× concentrated stocks; 10× TE -0.1 M Tris-HCl, 0.01 M EDTA, pH 7.5; 10× LiOAc-1 M LiOAc adjusted to pH 7.5 with dilute acetic acid). For each transformation, ˜5 μg of DNA was mixed with 70 μg of freshly denatured salmon sperm DNA (10 mg/mL, boiled for 20 min in a water bath, then chilled in ice/water) and 200 μL cells in TE/LiOAc were added and carefully mixed. Immediately, 1,200 μL of freshly prepared sterile 40% PEG 4,000 (prepared from stock solutions: 50% PEG 4000, 10× TE, 10× LiOAc, 8:1:1 v/v, pH 7.5) were added and carefully mixed. Cells were incubated for 30 min at 30° C. with constant agitation. Cells were incubated for 15 min at 42° C. and then collected by centrifugation (4,000 g, 1 min). Cells were resuspended in 200 μL YPD and plated onto selective plates. Plates were incubated at 30° C. until colonies appeared.

### Example 3

### Cellulose treatment

All chemicals, media components and supplements were of analytical grade standard. Phosphoric acid-swollen cellulose (PASC) was prepared as described by Den Haan et al., (2007). Briefly, Avicel® PH-101 (Fluka) (2 g) was first soaked with 6 mL of distilled water. Then, 50 mL of 86.2% phosphoric acid was added slowly to the tube and mixed well, followed by another 50 mL of phosphoric acid and mixing. The transparent solution was kept at 4° C. overnight to completely solubilize the cellulose, until no lumps remained in the reaction mixture. Next, 200 mL of ice-cold distilled water was added to the tube and mixed, followed by another 200 mL of water and mixing. The mixture was centrifuged at 3,500 rpm for 15 min and the supernatant was removed. Addition of distilled water and subsequent centrifugation were repeated. Finally, 10 mL of 2M sodium carbonate and 450 mL of water were added to the cellulose, followed by 2 or 3 washes with distilled water, until a final pH of 5-7 was obtained. Acid treatment of Whatman® Paper #1 was done as described above for Avicel®, except only 1 g of shredded paper was used.

### Example 4

### Yeast Fermentation

Single colonies were inoculated into 10 mL of media with appropriate supplements and with 2% glucose as a carbon source and incubated aerobically for 24-72 hours at 30° C. Yeast cells were collected by centrifugation for 10 min at 4,000 rpm and resuspended in 100 mL of media with 2% glucose. After incubation under aerobic conditions for 24-72 hours at 30° C. cells were harvested by centrifugation and washed with distilled water twice. Cell pellets were inoculated in 10 mL of media with either 2% glucose, or 200 g/L PASC or treated Whatman® Paper and ethanol fermentations were anaerobically performed at 30° C. in 15 mL tubes with closed caps. 0.2 mL aliquots were collected at different time points and analyzed using gas chromatography for ethanol concentration.

### Example 5

### Gas Chromatography Analysis

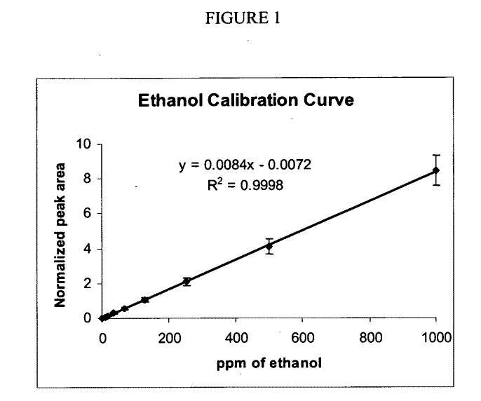

Fermentation products, such as ethanol, were analyzed using gas chromatography (GC) (5890 Series II Agilent Technologies, Wilmington, Del.) provided with a RTX-5 capillary column (30 m×0.53 mm i.d.×1.5 μm) (Restek, Bellefonte, Pa.) and flame ionization detection. Prior to analysis, the samples were centrifuged at 14,000×rpm for 10 minutes. The samples were diluted 20-fold with a 25 ppm aqueous solution of n-propanol as an internal standard. Helium was used as a carrier gas at 5 mL/min and was split 1 to 20 before the capillary column. Following sample injection, the column was held at 40° C. for 4 minutes and then ramped to 130° C. at a rate of 30° C./min. The GC was equipped with a 7673B auto-sampler (Agilent Technologies) and data were collected through contact closures and analyzed using Peak Simple software (SRI Instruments Torrance, Calif.). Linear calibration curves were developed for ethanol covering the range of 1000 ppm to 0.8 ppm. FIG. 1 is an example of a calibration curve for ethanol.

### Example 6

### Ethanol from Cellulose by Recombinant Yeast

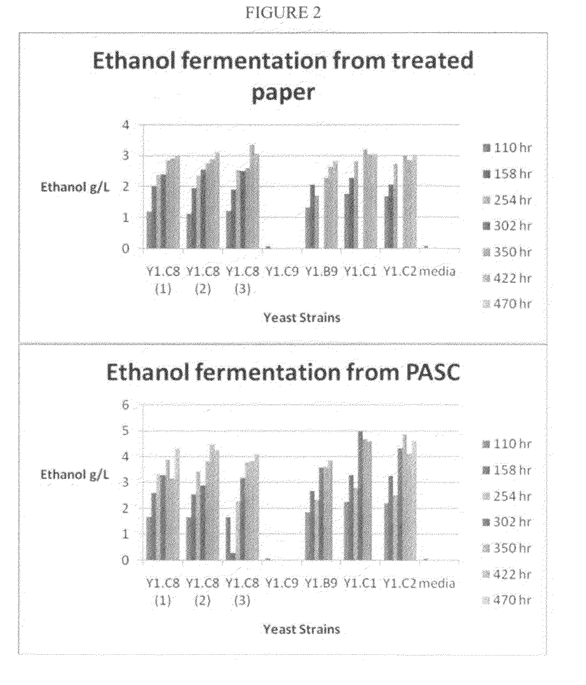

Several yeast strains were constructed for production of ethanol from cellulose. To ferment cellulose to ethanol, strains were constructed that codisplay three cellulases (EGII, CHBII and BGLI) on the yeast cell wall surface (Table 1). Furthermore, a second set of strains that produce secreted forms of the same cellulases was developed (Table 1). The strains with surface displayed cellulases and the strains expressing secreted cellulases are efficient hosts for the production of ethanol from either PASC or treated paper (FIG. 2). FIG. 2 illustrates fermentation of cellulose to ethanol by the above yeast strains. Fermentations were performed in 15 mL tubes with 10 mL of minimal media and 200 g/L PASC or treated Whatman® Paper. PASC, an amorphous type of cellulose, was prepared from Avicel® by treatment with phosphoric acid as described above in Example 3. Avicel® is a commercially available, crystalline form of cellulose produced by acid reflux hydrolysis of wood. Several independent recombinant yeast strains were used for each fermentation experiment. Yeast strains transformed with empty vectors, i.e., without cellulases genes, were used as negative controls. Yields in excess of 4 grams per liter of ethanol are demonstrated.

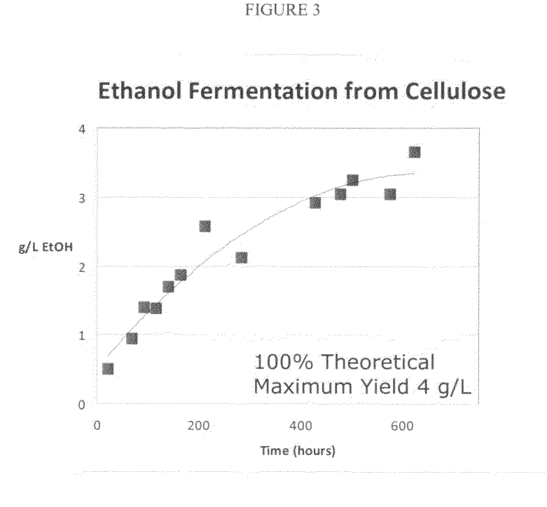

FIG. 3 shows ethanol production by yeast strain Y1.C8, as a function of time, from a 10 mL fermentation of PASC. Fermentations were performed at 30° C. with a starting concentration of PASC of 261 g/L. Remarkably, the ethanol producing yeast strains depolymerized cellulose and fermented it to ethanol with almost 100% of the maximum theoretical yield. At the peak of fermentation, 3.65 g/L of ethanol is produced representing conversion efficiency of 91.25%.

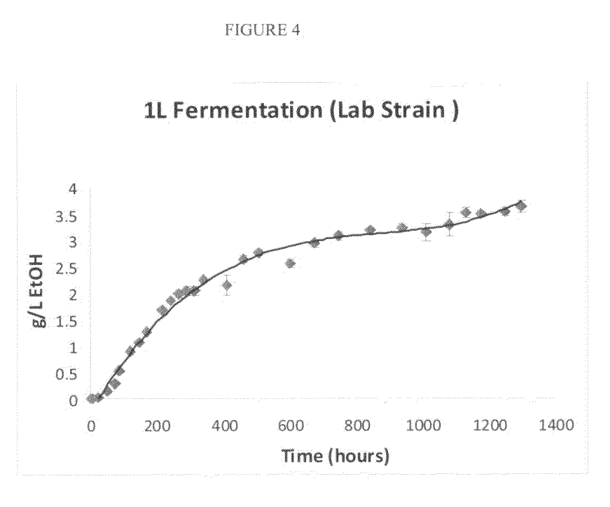

FIG. 4 shows ethanol production by yeast strain Y1.C1, as a function of time, from a one liter fermentation of treated paper. The data in this graph are the average of three samples taken at each time point, and the error bars are±one standard deviation.

### Example 7

### Expression of Xylose Assimilation Enzymes in S. cerevisiae

Xylose fermenting S. cerevisiae strains were also engineered. Wild-type strains of S. cerevisiae cannot utilize pentoses, such as xylose. However efficient fermentation of pentose sugars is advantageous to attain economically feasible processes for ethanol production from lignocellulosic biomass, as xylose could be 25%-30% of the fermentable carbon substrate depending on the feedstock. Anaerobic xylose fermentation by S. cerevisiae was first demonstrated by heterologous expression of xylose reductase (XR) and xylitol dehydrogenase (XDH) from Pichia stipitis together with overexpression of the endogenous xylulokinase (XK) (Ho et al., 1998, 1999). Alcohol fermentation from xylose was also performed by a recombinant S. cerevisiae strain carrying only one heterologous xylose isomerase (XI) gene from the fungus Piromyces sp. (Kuyper et al., 2003).

The open reading frame encoding XI (GenBank accession number AJ249909) was synthesized by Blue Heron Bio. Sites for restriction endonucleases SalI and KpnI were introduced at 5′- and 3′-ends of DNA, respectively. The sites for restriction endonucleases HindIII and KpnI were removed via one nucleotide substitutions that do not change the amino acid sequences. The synthesized DNA was cloned into the Blue Heron pUC119 vector. The sequence of the vector insert is shown below:

pUC119-AF105 (xylose isomerase (XI) construct):

(SEQ ID NO: 4)

GTCGACATGGCTAAGGAATATTTCCCACAAATTCAAAAGATTAAGTTCGA
AGGTAAGGATTCTAAGAATCCATTAGCCTTCCACTACTACGATGCTGAAA
AGGAAGTCATGGGTAAGAAAATGAAGGATTGGTTACGTTTCGCCATGGCC
TGGTGGCACACTCTTTGCGCCGAAGGTGCTGACCAATTCGGTGGAGGTAC
AAAGTCTTTCCCATGGAACGAAGGTACTGATGCTATTGAAATTGCCAAGC
AAAAGGTTGATGCTGGTTTCGAAATCATGCAAAAACTTGGTATTCCATAC
TACTGTTTCCACGATGTTGATCTTGTTTCCGAAGGTAACTCTATTGAAGA
ATACGAATCCAACCTTAAGGCTGTCGTTGCTTACCTCAAGGAAAAGCAAA
AGGAAACCGGTATTAAACTTCTCTGGAGTACTGCTAACGTCTTCGGTCAC
AAGCGTTACATGAACGGTGCCTCCACTAACCCAGACTTTGATGTTGTCGC
CCGTGCTATTGTTCAAATTAAGAACGCCATAGACGCCGGTATTGAACTTG
GTGCTGAAAACTACGTCTTCTGGGGTGGTCGTGAAGGTTACATGAGTCTC
CTTAACACTGACCAAAAGCGTGAAAAGGAACACATGGCCACTATGCTTAC
CATGGCTCGTGACTACGCTCGTTCCAAGGGATTCAAGGGTACTTTCCTCA
TTGAACCAAAGCCAATGGAACCAACCAAGCACCAATACGATGTTGACACT
GAAACCGCTATTGGTTTCCTTAAGGCCCACAACTTAGACAAGGACTTCAA
GGTCAACATTGAAGTTAACCACGCTACTCTTGCTGGTCACACTTTCGAAC
ACGAACTTGCCTGTGCTGTTGATGCTGGTATGCTCGGTTCCATTGATGCT
AACCGTGGTGACTACCAAAACGGTTGGGATACTGATCAATTCCCAATTGA
TCAATACGAACTCGTCCAAGCATGGATGGAAATCATCCGTGGTGGTGGTT
TCGTTACTGGTGGAACCAACTTCGATGCCAAGACTCGTCGTAACTCTACT
GACCTCGAAGACATCATCATTGCCCACGTTTCTGGTATGGATGCTATGGC
TCGTGCTCTTGAAAACGCTGCCAAGCTCCTCCAAGAATCTCCATACACCA
AGATGAAGAAGGAACGTTACGCTTCCTTCGACAGTGGTATTGGTAAGGAC
TTTGAAGATGGTAAGCTCACCCTCGAACAAGTTTACGAATACGGTAAGAA
GAACGGTGAACCAAAGCAAACTTCTGGTAAGCAAGAACTCTACGAAGCTA
TTGTTGCCATGTACCAATAAGGTACC

### Where Nucleotides:

1 to 6 is a SalI restriction site;
7 to 1317 is xylose isomerase from Pyromyces sp. (GenBank accession number AJ249909), with the following nucleotide changes introduced (numbering according to the AJ249909 DNA sequence): G283A; G415A; T970A and T112A;
1318 to 1320 is a TAA STOP codon; and
1321 to 1326 is a KpnI restriction site.

The resulting plasmid, pUC119-AF105, was digested with SalI-KpnII and the 1326 bp DNA fragment was gel purified. The purified DNA fragment was ligated into the SalI-KpnI digested vector YEplac195-AF101-at to generate plasmid pYEplac195-AF105. This plasmid was used for the transformation of yeast cells as well as for cotransformation of cells already containing cellulase genes as described above. Alternatively, xylose fermentation by S. cerevisiae can be achieved using heterologous expression of xylose reductase (XR) and xylitol dehydrogenase (XDH) from Pichia stipitis together with overexpression of the endogenous xylulokinase (XK).

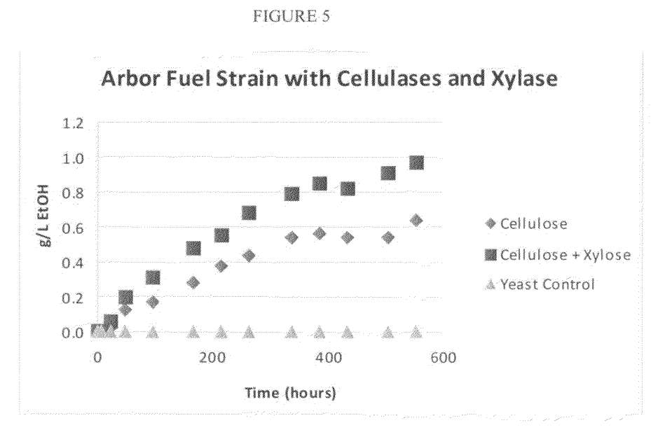

FIG. 5 illustrates the fermentation of ethanol from cellulose and xylose using the strains described above. Fermentations were performed in 15 mL tubes with 10 mL of minimal media and 100 g/L PASC or 100 g/L PASC with 1% xylose. The amount of ethanol produced from the xylose containing solutions is higher than that produced from PASC alone demonstrating the recombinant yeast's utilization of both the cellulose and the xylose.

### Example 8

### Construction of Integrating Vectors Encoding Cellulase Genes

In order to create stable transformants in yeast strains suitable for industrial fermentations, which avoid the requirement for constant selection pressure, the cellulases described in Example 1 were also inserted into the genomic DNA of S. cervisiae. In this approach, the need for auxotrophic markers was eliminated. Instead of using auxotrophic markers, the transformants were selected by using dominant resistant-selectable markers (typically antibiotics). Because expression vectors for industrial strains are not commercially available, an expression vector AFV1 (pYI-kanMX-18SrDNA) was created. This vector has the G418 (antibiotic) resistance gene, KanMX. To integrate multiple copies of the cellulase genes into the chromosomes of Saccharomyces cerevisiae, a fragment of 18S rDNA from S. cerevisiae was also inserted in AFV1, which results in the integration of copies of target genes into the yeast's multiple 18S sites.

The three cellulase genes were cloned into a single vector, AFV1. AFV1 was created from the integrating vector YIplac211 as follows: The KanMX gene was amplified by PCR from the pUG6 plasmid using primers 1.H9 CCTTAGCGGCGCCAGCTGATGCTTCGTACGCTGCAG (SEQ ID NO: 5) and 1.H10 CCTTAGCAGGCCTGCATAGGCCACTAGTGGATCTTATATC (SEQ ID NO: 6). The resulting PCR product was digested with NarI and StuI and inserted into the vector YIplac211 also digested with NarI and StuI. The resulting vector was named YI-KanMX. Then 18S rDNA was amplified by PCR from genomic yeast DNA using primers 1.I1 CCTTAGCGACGTCTAATGATCCTTCCGCAGG (SEQ ID NO: 7) and 1.I2 CCTTAGCGATATCTATCTGGTTGATCCTGCCAG (SEQ ID NO: 8). The resulting PCR product was digested with AatII and EcoRV and inserted into the vector YI-KanMX also digested with AatII and EcoRV. The resulting vector was named YI-KanMX-18SrDNA. The ampicillin resistance gene from YI-KanMX-18srDNA was removed by PCR amplification of the entire vector DNA using primers 2.A7 TACCAGCTTAAGTTTCACTCCTAGGCAAATAGGGGTTCCGCGCACATTTCC (SEQ ID NO: 9) and 2.A8 CATAAATGCGGCCGCTACCTAGTTTAAACAGGATCTAGGTGAAGATCCTTTTTGATA ATC (SEQ ID NO: 10). The resulting vector was named AFV 1. Primers 2.A7 and 2.A8 also introduced several unique restriction sites (AvrII, AflII, NotI and PmeI) that were used for cloning of three secreted cellulases. Insertion of the cellulase genes into AFV1 was done by procedure similar to that described into Example 1.

### Example 9

### Construction of Recombinant Industrial Yeast Strains with Integrated Cellulases

Two industrial diploid yeast strains, a distillery strain—SuperStart (White Labs, Boulder, Colo.) and a wine strain—K1-V1116 (Lallemand, Montreal, QC, Canada) were transformed with cellulase genes. SuperStart is intended for use in fuel ethanol and beverage alcohol fermentations. It ferments well at temperatures up to 93° F. (34° C.) and in a pH range of 3.5 to 6.0. The K1-V1116 is a vigorously fermenting, dominant strain that will overcome wild yeast, and ferments well in a must that is low in nutrients. Transformation of the yeast cells was done by electroporation using linear DNA. The AFVI vector containing three cellulases gene was linearized by digesting 10 μg plasmid DNA with 100 units of NheI enzyme, which cuts the vector only once in the 18SrDNA region. Transformation mixtures were plated on YPD agar dishes containing various concentrations of G418 (up to 10 mg/mL). Only recombinant yeast strains with integrated genes can grow on agar dishes containing G418 antibiotic.

Remarkably, both SuperStart and K1-V1116 strains were unable to form complete tetrads after sporulation and dissection. The SuperStart spores germinated very poorly, forming a few very small colonies. By contrast, segregation of viability of the K1-V1116 spores was close to a 2:2 ratio suggesting that this strain has a single copy mutation (i.e., the strain is a heterozygous diploid) in a gene essential for germination or viability. To construct a genetically tractable yeast strain useful for subsequent genetic manipulations, cells derived from viable spores were resporulated and asci were dissected. The resultant spores showed 100% viability, demonstrating that this strain can be subjected to further genetic analysis and manipulation.

### Example 10

### Ethanol from Cellulose by Recombinant Industrial Yeast Strains with Integrated Cellulases

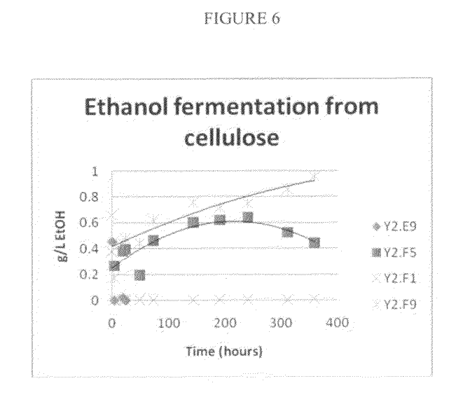

FIG. 6 illustrates the fermentation of ethanol from cellulose using the strains described in Example 9 above. Fermentations were performed in 15 mL tubes with 10 mL of minimal media and 40% PASC. Several independent recombinant yeast strains were used for each fermentation experiment. We found that several yeast strains (Y2.F5 and Y2.F9) with integrated cellulases genes fermented cellulose to ethanol. The unmodified industrial yeast strains (i.e., SuperStart—Y2.E9 and K1-V1116—Y2.F9) are not capable of fermenting ethanol from cellulose.

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

TABLE 1
Yeast strains and plasmids used
Yeast strains

| | |
|---|---|
| AFY1 | MATα his3-Δ200 leu_3,112 ura3-52 |
| | lys2-801 trp1-1 |
| Y1.C8 | AFYI with three attached cellulases |
| Y1.B9 | AFYI with three secreted cellulases |
| Y1.C1 | AFYI with three secreted cellulases |
| Y1.C2 | AFYI with three secreted cellulases |
| Y2.F2 | SuperStart with three secreted cellulases |
| Y2.F3 | SuperStart with three secreted cellulases |
| Y2.F4 | SuperStart with three secreted cellulases |
| Y2.F5 | SuperStart with three secreted cellulases |
| Y2.F6 | SuperStart with three secreted cellulases |
| Y2.F7 | SuperStart with three secreted cellulases |
| Y2.F8 | K1-V1116 with three secreted cellulases |
| Y2.F9 | K1-V1116 with three secreted cellulases |
| Y2.G1 | K1-V1116 with three secreted cellulases |
| Y2.G2 | K1-V1116 with three secreted cellulases |
| Y2.G3 | K1-V1116 with three secreted cellulases |
| Y2.G4 | K1-V1116 with three secreted cellulases |

Plasmids

| | |
|---|---|
| pUC119-AF101 | cellobiohydrolase II (CBHII) construct |
| pUC119-AF105 | xylose isomerase (XI) construct |
| YEplac112-AF101-at | expression construct with attached CBHII |
| YEplac181-AF101-at | expression construct with attached CBHII |
| YEplac195-AF101-at | expression construct with attached CBHII |
| YEplac181-AF102-at | expression construct with attached BGLI |
| YEplac112-AF103-at | expression construct with attached EGII |
| YEplac195-AF101-sec | expression construct with secreted CBHII |
| YEplac181-AF102-sec | expression construct with secreted BGLI |
| YEplac112-AF103-sec | expression construct with secreted EGII |
| YEplac195-AF105 | expression construct with XI |
| AFV1-AF101-sec-- | expression construct for integration of three |
| AF102-sec-AF103-sec | secreted cellulases into the 18S rDNA |
| | region of yeast genome |

### REFERENCES

Ausubel F M, Brent R, Kingston R E, More D D, Seidman J G, Smith J A, Struhl K. 2002. Short Protocols in Molecular Biology. John Wiley and Sons, New York.
Den Haan R, Rose S H, Lynd L R, van Zyl W H. 2007. Hydrolysis and fermentation of amorphous cellulose by recombinant Saccharomyces cerevisiae. Metab Eng 9:87-94.
Durre P, Kuhn A, Gottwald M, Gottschalk. 1987. Enzymatic investigations on butanol dehydrogenase and butyraldehyde dehydrogenase in extracts of Clostridium acetobutylicum. Appl Microbiol Biotechnol 26:268-272.
Fujita Y, Ito J, Ueda M, Fukuda H, Kondo A. 2004. Synergistic saccharification, and direct fermentation to ethanol, of amorphous cellulose by use of an engineered yeast strain codisplaying three types of cellulolytic enzyme. Appl Environ Microbiol 70:1207-1212.
Gietz R D, Sugino A. 1988. New yeast-Escherichia coli shuttle vectors constructed with in vitro mutagenized yeast genes lacking six-base pair restriction sites. Gene 74:527-534.

Guldener U, Heck S, Fiedler T, Beinhauer J, Heenmann J. 1996. A new efficient gene disruption cassette for repeated use in budding yeast. Nucleic Acid Research 24:2519-2524.
Hammel K E, Cullen D. 2008. Role of fungal peroxidases in biological ligninolysis. Curr Opin Plant Biol 11: 349-355.
Hartmanis M G, Gatenbeck S. 1984. Intermediary Metabolism in Clostridium acetobutylicum: Levels of Enzymes Involved in the Formation of Acetate and Butyrate. Appl Environ Microbiol 47:1277-1283.
Ho N W, Chen Z, Brainard A P. 1998. Genetically engineered Saccharomyces yeast capable of effective cofermentation of glucose and xylose. Appl Environ Microbiol 64:1852-1859.
Ho N W, Chen Z, Brainard A P, Sedlak M. 1999. Successful design and development of genetically engineered Saccharomyces yeasts for effective co-fermentation of glucose and xylose from cellulosic biomass to fuel ethanol. Adv Biochem Eng Biotechnol 65:163-192.
Jonsson L J, Palmqvist E, Nilvebrant N O, Hahn-Hagerdal B. 1998. Detoxifcation of wood hydrolysates with laccase and peroxidase from the white-rot fungus Trametes versicolor. Appl Microbiol Biotechnol 49:691-697.
Kuyper M, Harhangi H R, Stave A K, Winkler A A, Jetten M S, de Laat W T, den Ridder J J, Op den Camp H J, van Dijken J P, Pronk J T. 2003. High level functional expression of a fungal xylose isomerase: the key to efficient ethanolic fermentation of xylose by Saccharomyces cerevisiae? FEMS Yeast Res 4:69-78.
Lehman T C, Hale D E, Bhala A, Thorpe C. 1990. An acyl-coenzyme A dehydrogenase assay utilizing the ferricenium ion. Anal Biochem 186:280-284.
Lynd L R, Weimer P J, van Zyl W H, Pretorius I S. 2002. Microbial cellulose utilization: fundamentals and biotechnology. Microbiol Mol Biol Rev 66:506-577.
Olsson L, Hahn-Hagerdal B. 1996. Fermentation of lignocellulosic hydrolysates for ethanol production. Enzyme Microb Technol 18:312-331.
Rodríguez-Couto S, Toca-Herrera J L. 2006. Industrial and biotechnological applications of laccases: a review. Biotechnol Adv 24: 500-513.
Sambrook J, Fritsch E F, Maniatis T. 1989. Molecular Cloning. A Laboratory Manual. Cold Spring Harbor Laboratory Press: Cold Spring Harbor, N.Y.
Sherman F, Fink G R, Hicks J B. 1986. Methods in Yeast Genetics. Cold Spring Harbor Laboratory Press: Cold Spring Harbor, N.Y.
van Zyl W H, Lynd L R, den Haan R, McBride J E. 2007. Consolidated bioprocessing for bioethanol production using Saccharomyces cerevisiae. Adv Biochem Eng Biotechnol 108:205-235
Wiesenborn D P, Rudolph F B, Papoutsakis E T. 1989. Coenzyme A transferase from Clostridium acetobutylicum ATCC 824 and its role in the uptake of acids. Appl Environ Microbiol 55:323-329.
Wolfenden R, Snider M J. 2001. The depth of chemical time and the powder of enzyme as catalysts. Acc Chem Res 34:938-945.
Zhang Y-H P, Lynd L R. 2004. Toward an aggregated understanding of enzymatic hydrolysis of cellulose: noncomplexed cellulose systems. Biotechnol Bioeng 88:797-824.
Zhang Y-H P, Himmel M E, Mielenz J R. 2006. Outook for cellulase improvement: screening and selection strategies. Biotechnol Adv 24:452-481.

What is claimed is:

1. A yeast strain, comprising: a yeast cell comprising heterologous genes that encode the cellulase enzymes: endoglucanase II, cellobiohydrolase II, and β-glucosidase I, wherein each of said genes encoding said cellulase enzymes are stably integrated into the yeast chromosome, wherein said cellulase enzymes are secreted external to the cell and wherein said yeast cell is capable of converting cellulose to ethanol.

2. The yeast strain of claim 1, wherein said yeast strain is an industrial yeast strain.

3. The yeast strain of claim 2, wherein said industrial yeast strain has a high tolerance for ethanol.

4. The yeast strain of claim 3, wherein said industrial yeast strain tolerates ethanol concentrations of about 18% or greater.

5. The yeast strain of claim 2, wherein said industrial yeast strain tolerates high temperatures.

6. The yeast strain of claim 5, wherein said industrial yeast strain tolerates temperatures of about 34° C. or greater.

7. The yeast strain of claim 6, wherein said industrial yeast strain tolerates temperatures of about 37° C. or greater.

8. The yeast strain of claim 2, wherein said industrial yeast strain has a high growth rate.

9. The yeast strain of claim 8, wherein said industrial yeast strain has a doubling time of about 90 minutes or less.

10. The yeast strain of claim 3, wherein said industrial yeast strain tolerates high temperatures.

11. The yeast strain of claim 10, wherein said industrial yeast strain has a high growth rate.

12. The yeast strain of claim 1, wherein multiple copies of each of the heterologous cellulase genes are stably integrated into the yeast chromosome.

13. The yeast strain of claim 1, wherein the yeast is a Saccharomyces species.

14. The yeast strain of claim 13, wherein said yeast is a member of a species selected from the group consisting of Saccharomyces carlsburgenesis, Saccharomyces bayanus, Saccharomyces cerevisiae and hybrids thereof.

15. The yeast strain of claim 14, wherein said yeast is a Saccharomyces cerevisiae.

16. The yeast strain of claim 1, wherein the endoglucanase II and cellobiohydrolase II genes are from T. reesei and the β-glucosidase I gene is from A. aculeatus.

17. The yeast strain of claim 16, wherein the endoglucanase II, cellobiohydrolase II and β-glucosidase I genes comprise nucleotides 7 to 1197 of SEQ ID NO: 3 nucleotides 785 to 2125 of SEQ ID NO: 1 and nucleotides 7 to 2529 of SEQ ID NO: 2, respectively.

18. The yeast strain of claim 17, wherein the endoglucanase II, cellobiohydrolase II and β-glucosidase I genes are operably linked to the GAPDH (glyceraldehyde 3-phosphate dehydrogenase) promoter and the CYC1 (cytochrome C) terminator.

19. The yeast strain of claim 18, wherein the GAPDH promoter and the CYC1 terminator comprise nucleotides 13 to 667 and nucleotides 3105 to 3356 of SEQ ID NO: 1, respectively.

20. The yeast strain of claim 1, wherein said yeast converts cellulose to ethanol at greater than 80% of the maximum theoretical yield.

21. The yeast strain of claim 20, wherein said yeast converts cellulose to ethanol at greater than 90% of the maximum theoretical yield.

22. The yeast strain of claim 21, wherein said yeast converts cellulose to ethanol at greater than 95% of the maximum theoretical yield.

23. The yeast strain of claim 22, wherein said yeast converts cellulose to ethanol at about 99% or greater of the maximum theoretical yield.

24. The yeast strain of claim 1, wherein said yeast converts cellulose to ethanol with a yield greater than 3 g/L.

25. The yeast strain of claim 24, wherein said yeast converts cellulose to ethanol with a yield greater than 3.2 g/L.

26. The yeast strain of claim 25, wherein said yeast converts cellulose to ethanol with a yield greater than 3.5 g/L.

27. The yeast strain of claim 26, wherein said yeast converts cellulose to ethanol with a yield greater than 3.8 g/L.

28. The yeast strain of claim 27, wherein said yeast converts cellulose to ethanol with a yield of about 4 g/L or greater.

29. A method for the production of ethanol from cellulose, comprising:
(a) providing a yeast strain of claim 1; and
(b) contacting the yeast with cellulose under conditions whereby ethanol is produced.

30. The method of claim 29, further comprising the step of isolating the ethanol that is produced.

31. A recombinant microorganism, comprising:
(1) at least one heterologous gene that encodes a cellulase enzyme; and
(2) at least one heterologous gene that encodes a polypeptide involved in the fermentation of a pentose sugar;
wherein said recombinant microorganism converts hemicellulose to ethanol.

32. The microorganism of claim 31, wherein said pentose sugar is xylose.

33. The microorganism of claim 32, wherein said polypeptide involved in the fermentation of xylose is a xylose isomerase.

34. The microorganism of claim 33, wherein the xylose isomerase gene is from Piromyces sp.

35. The microorganism of claim 32, wherein the microorganism comprises heterologous genes that encode a xylose reductase and a xylitol dehydrogenase.

36. The microorganism of claim 35, wherein the xylose reductase and xylitol dehydrogenase genes are from Pichia stipitis.

37. A method for the production of ethanol from hemicellulose, comprising:
(a) providing a recombinant microorganism according to claim 31; and
(b) contacting the microorganism with hemicellulose under conditions whereby ethanol is produced.

38. The method of claim 37, further comprising the step of isolating the ethanol that is produced.


SEQUENCE LISTING (10 sequences)

SEQ ID NO: 1 - DNA, 3368 bp, Artificial Sequence
  Description of Artificial Sequence Synthetic construct
aagcttgcatgcagtttatcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacggatctgcaggtcgacatgcaactgttcaatttgccattgaaagtttcattctttctcgtcctctcttacttttctttgctcgtttctgctgactacaaggacgatgacgacaaatctagacaggcttgctcaagcgtctggggccaatgtggtggccagaattggtcgggtccgacttgctgtgcttccggaagcacatgcgtctactccaacgactattactcccagtgtcttcccggcgctgcaagctcaagctcgtccacgcgcgccgcatcgacgacttcacgagtatcccccacaacatcccggtcgagttccgcgacgcctccacctggttctactactaccagagtacctccagtcggatcgggaaccgctacgtattcaggcaacccttttgttggggtcactccttgggccaatgcatattacgcctctgaagttagcagcctcgctattcctagcttgactggagccatggccactgccgcagcagctgtcgcaaaggttccctcttttatgtggctagatactcttgacaagacccctctcatggagcaaaccttggccgacatccgcaccgccaacaagaatggcggtaactatgccggacagtttgtggtgtatgacttgccggatcgcgattgcgctgcccttgcctcgaatggcgaatactctattgccgatggtggcgtcgccaaatataagaactatatcgacaccattcgtcaaattgtcgtggaatattccgatatccggaccctcctggttattgagcctgactctcttgccaacctggtgaccaacctcggtactccaaagtgtgccaatgctcagtcagcctaccttgagtgcatcaactacgccgtcacacagctgaaccttccaaatgttgcgatgtatttggacgctggccatgcaggatggcttggctggccggcaaaccaagacccggccgctcagctatttgcaaatgtttacaagaatgcatcgtctccgagagcacttcgcggattggcaaccaatgtcgccaactacaacgggtggaacattaccagccccccatcgtacacgcaaggcaacgctgtctacaacgagaagctgtacatccacgctattggacgtcttcttgccaatcacggctggtccaacgccttcttcatcactgatcaaggtcgatcgggaaagcagcctaccggacagcaacagtggggagactggtgcaatgtgatcggcaccggatttggtattcgcccatccgcaaacactggggactcgttgctggattcgtttgtctgggtcaagccaggcggcgagtgtgacggcaccagcgacagcagtgcgccacgatttgactcccactgtgcgctcccagatgccttgcaaccggcgcctcaagctggtgcttggttccaagcctactttgtgcagcttctcacaaacgcaaacccatcgttcctgggatccagcgccaaaagctcttttatctcaaccactactactgatttaacaagtataaacactagtgcgtattccactggttccatttccacagtagaaacaggcaatcgaactacatcagaagtgatcagtcatgtggtgactaccagcacaaaactgtctccaactgctactaccagcctgacaattgcacaaaccagtatctattctactgactcaaatatcacagtaggaacagatattcacaccacatcagaagtgattagtgatgtggaaaccattagcagagaaacagcttcgaccgttgtagccgctccaacctcaacaactggatggacaggcgctatgaatacttacatcccgcaatttacatcctcttctttcgcaacaatcaacagcacaccaataatctcttcatcagcagtatttgaaacctcagatgcttcaattgtcaatgtgcacactgaaaatatcacgaatactgctgctgttccatctgaagagcccacttttgtaaatgccacgagaaactccttaaattccttttgcagcagcaaacagccatccagtccctcatcttatacgtcttccccactcgtatcgtccctctccgtaagcaaaacattactaagcaccagttttacgccttctgtgccaacatctaatacatatatcaaaacggaaaatacgggttactttgagcacacggctttgacaacatcttcagttggccttaattcttttagtgaaacagcactctcatctcagggaacgaaaattgacacctttttagtgtcatccttgatcgcatatccttcttctgcatcaggaagccaattgtccggtatccaacagaatttcacatcaacttctctcatgatttcaacctatgaaggtaaagcgtctatatttttctcagctgaactcggttcgatcatttttctgcttttgtcgtacctgctattctaacccgggtacctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccgagctcgaattc

SEQ ID NO: 2 - DNA, 2544 bp, Artificial Sequence
  Description of Artificial Sequence Synthetic construct
tctagagatgaactggcgttctctcctcctttctacccctctccgtgggccaatggccagggagagtgggcggaagcctaccagcgtgcagtggccattgtatcccagatgactctggatgagaaggtcaacctgaccaccggaactggatgggagctggagaagtgcgtcggtcagactggtggtgtcccaagactgaacatcggtggcatgtgtcttcaggacagtcccttgggtattcgtgatagtgactacaattcggctttccctgctggtgtcaacgttgctgcgacatgggacaagaaccttgcttatctacgtggtcaggctatgggtcaagagttcagtgacaaaggaattgatgttcaattgggaccggccgcgggtcccctcggcaggagccctgatggaggtcgcaactgggaaggtttctctccagacccggctcttactggtgtgctctttgcggagacgattaagggtattcaagacgctggtgtcgtggcgacagccaagcattacattctcaatgagcaagagcatttccgccaggtcgcagaggctgcgggctacggattcaatatctccgacacgatcagctctaacgttgatgacaagaccattcatgaaatgtacctctggcccttcgcggatgccgttcgcgccggcgttggcgccatcatgtgttcctacaaccagatcaacaacagctacggttgccagaacagttacactctgaacaaacttctgaaggccgaactcggcttccagggctttgtgatgtctgactggggtgctcaccacagtggtgttggctctgctttggccggcttggatatgtcaatgcctggcgatatcaccttcgattctgccactagtttctggggaaccaacctgaccattgctgtgctcaacggaaccgtcccgcagtggcgcgttgacgacatggctgtccgtatcatggctgcctactacaaggttggccgcgaccgcctgtaccagccgcctaacttcagctcctggactcgcgatgaatacggcttcaagtatttctacccccaggaagggccctatgagaaggtcaatcactttgtcaatgtgcagcgcaaccacagcgaggttattcgcaagttgggagcagacagtactgttctactgaagaacaacaatgccctgccgctgaccggaaaggagcgcaaagttgcgatcctgggtgaagatgctggttccaactcgtacggtgccaatggctgctctgaccgtggctgtgacaacggtactcttgctatggcttggggtagcggcactgccgaatttccatatctcgtgacccctgagcaggctattcaagccgaggtgctcaagcataagggcagcgtctacgccatcacggacaactgggcgctgagccaggtggagaccctcgctaaacaagccagtgtctctcttgtatttgtcaactcggacgcgggagagggctatatctccgtggacggaaacgagggcgaccgcaacaacctcaccctctggaagaacggcgacaacctcatcaaggctgctgcaaacaactgcaacaacaccatcgttgtcatccactccgttggacctgttttggttgacgagtggtatgaccaccccaacgttactgccatcctctgggcgggcttgcctggccaggagtctggcaactccttggctgacgtgctctacggccgcgtcaacccaggcgccaaatctccattcacctggggcaagacgagggaggcgtacggggattaccttgtccgtgaactcaacaacggcaacggagcaccccaagatgatttctcggaaggtgttttcattgactaccgcggattcgacaagcgcaatgagaccccgatctacgagttcggacatggtctgagctacaccactttcaactactctggccttcacatccaggttctcaacgcttcctccaacgctcaagtagccactgagactggcgccgctcccaccttcggacaagtcggcaatgcctctgactacgtgtaccctgagggattgaccagaatcagcaagttcatctatccctggcttaattccacagacctgaaggcctcatctggcgacccgtactatggagtcgacaccgcggagcacgtgcccgagggtgctactgatggctctccgcagcccgttctgcctgccggtggtggctctggtggtaacccgcgcctctacgatgagttgatccgtgtttcggtgacagtcaagaacactggtcgtgttgccggtgatgctgtgcctcaattgtatgtttcccttggtggacccaatgagcccaaggttgtgttgcgcaaattcgaccgcctcaccctcaagccctccgaggagacggtgtggacgactaccctgacccgccgcgatctgtctaactgggacgttgcggctcaggactgggtcatcacttcttacccgaagaaggtccatgttggtagctcttcgcgtcagctgccccttcacgcggcgctcccgaaggtgcaaggatcctaaggtacc

SEQ ID NO: 3 - DNA, 1212 bp, Artificial Sequence
  Description of Artificial Sequence Synthetic construct
tctagacagcagactgtctggggccagtgtggaggtattggttggagcggacctacgaattgtgctcctggctcagcttgttcgaccctcaatccttattatgcgcaatgtattccgggagccactactatcaccacttcgacccggccaccatccggtccaaccaccaccaccagggctacctcaacaagctcatcaactccacccactagctctggggtccgatttgccggcgttaacatcgcgggttttgactttggctgtaccacagatggcacttgcgttacctcgaaggtttatcctccgttgaagaacttcaccggctcaaacaactaccccgatggcatcggccagatgcagcacttcgtcaacgaggacgggatgactattttccgcttacctgtcggatggcagtacctcgtcaacaacaatttgggcggcaatcttgattccacgagcatttccaagtatgatcagcttgttcaggggtgcctgtctctgggcgcatactgcatcgttgacatccacaattatgctcgatggaacggtgggatcattggtcagggcggccctactaatgctcaattcacgagcctttggtcgcagttggcatcaaagtacgcatctcagtcgagggtgtggttcggcatcatgaatgagccccacgacgtgaacatcaacacctgggctgccacggtccaagaggttgtaaccgcaatccgcaacgctggtgctacgtcgcaattcatctctttgcctggaaatgattggcaatctgctggggctttcatatccgatggcagtgcagccgccctgtctcaagtcacgaacccggatgggtcaacaacgaatctgatttttgacgtgcacaaatacttggactcagacaactccggtactcacgccgaatgtactacaaataacattgacggcgccttttctccgcttgccacttggctccgacagaacaatcgccaggctatcctgacagaaaccggtggtggcaacgttcagtcctgcatacaagacatgtgccagcaaatccaatatctcaaccagaactcagatgtctatcttggctatgttggttggggtgccggatcatttgatagcacgtatgtcctgacggaaacaccgactggcagtggtaactcatggacggacacatccttggtcagctcgtgtctcgcaagaaagggatcctaaggtacc

SEQ ID NO: 4 - DNA, 1326 bp, Artificial Sequence
  Description of Artificial Sequence Synthetic construct
gtcgacatggctaaggaatatttcccacaaattcaaaagattaagttcgaaggtaaggattctaagaatccattagccttccactactacgatgctgaaaaggaagtcatgggtaagaaaatgaaggattggttacgtttcgccatggcctggtggcacactctttgcgccgaaggtgctgaccaattcggtggaggtacaaagtctttcccatggaacgaaggtactgatgctattgaaattgccaagcaaaaggttgatgctggtttcgaaatcatgcaaaaacttggtattccatactactgtttccacgatgttgatcttgtttccgaaggtaactctattgaagaatacgaatccaaccttaaggctgtcgttgcttacctcaaggaaaagcaaaaggaaaccggtattaaacttctctggagtactgctaacgtcttcggtcacaagcgttacatgaacggtgcctccactaacccagactttgatgttgtcgcccgtgctattgttcaaattaagaacgccatagacgccggtattgaacttggtgctgaaaactacgtcttctggggtggtcgtgaaggttacatgagtctccttaacactgaccaaaagcgtgaaaaggaacacatggccactatgcttaccatggctcgtgactacgctcgttccaagggattcaagggtactttcctcattgaaccaaagccaatggaaccaaccaagcaccaatacgatgttgacactgaaaccgctattggtttccttaaggcccacaacttagacaaggacttcaaggtcaacattgaagttaaccacgctactcttgctggtcacactttcgaacacgaacttgcctgtgctgttgatgctggtatgctcggttccattgatgctaaccgtggtgactaccaaaacggttgggatactgatcaattcccaattgatcaatacgaactcgtccaagcatggatggaaatcatccgtggtggtggtttcgttactggtggaaccaacttcgatgccaagactcgtcgtaactctactgacctcgaagacatcatcattgcccacgtttctggtatggatgctatggctcgtgctcttgaaaacgctgccaagctcctccaagaatctccatacaccaagatgaagaaggaacgttacgcttccttcgacagtggtattggtaaggactttgaagatggtaagctcaccctcgaacaagtttacgaatacggtaagaagaacggtgaaccaaagcaaacttctggtaagcaagaactctacgaagctattgttgccatgtaccaataaggtacc

SEQ ID NO: 5 - DNA, 36 bp, Artificial Sequence
  Description of Artificial Sequence Synthetic primer
ccttagcggcgccagctgatgcttcgtacgctgcag

SEQ ID NO: 6 - DNA, 40 bp, Artificial Sequence
  Description of Artificial Sequence Synthetic primer
ccttagcaggcctgcataggccactagtggatcttatatc

SEQ ID NO: 7 - DNA, 31 bp, Artificial Sequence
  Description of Artificial Sequence Synthetic primer
ccttagcgacgtctaatgatccttccgcagg

SEQ ID NO: 8 - DNA, 33 bp, Artificial Sequence
  Description of Artificial Sequence Synthetic primer
ccttagcgatatctatctggttgatcctgccag

SEQ ID NO: 9 - DNA, 51 bp, Artificial Sequence
  Description of Artificial Sequence Synthetic primer
taccagcttaagtttcactcctaggcaaataggggttccgcgcacatttcc

SEQ ID NO: 10 - DNA, 60 bp, Artificial Sequence
  Description of Artificial Sequence Synthetic primer
cataaatgcggccgctacctagtttaaacaggatctaggtgaagatcctttttgataatc